(19) United States
(12) Patent Application Publication
(10) Pub. No.: US 2008/0206184 A1
(43) Pub. Date: Aug. 28, 2008

(54) Chemically Modified Protein Compositions and Methods

(75) Inventors: Colin V. Gegg, Newbury Park, CA (US); Olaf B. Kinstler, Newbury Park, CA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMGEN INC., Thousand Oaks, CA (US)

(21) Appl. No.: 12/027,641

(22) Filed: Feb. 7, 2008

Related U.S. Application Data
(63) Continuation of application No. 11/088,365, filed on Mar. 23, 2005, now abandoned.
(60) Provisional application No. 60/555,792, filed on Mar. 23, 2004.

(51) Int. Cl. A61K 31/74; C08G 65/32

(52) U.S. Cl. 424/78.17; 525/403

(57) ABSTRACT

The present invention broadly relates to the field of protein modification and more specifically, water soluble block polymers, their attachment to drugs, and methods of making and use thereof.

This application claims the benefit of U.S. Provisional Application No. 60/555,792, filed Mar. 23, 2004, which is hereby incorporated by reference.

### FIELD OF THE INVENTION

The present invention broadly relates to the field of protein modification and more specifically, water soluble block polymers, their attachment to drugs, and methods of making and use thereof.

### BACKGROUND

Conjugation of a water soluble polymer to a drug can provide an enhancement of the solubility, durability and effectiveness of the molecule. For example, when a protein is conjugated to polymer, the modification may inhibit proteolysis by blocking physical contact through steric interactions and preventing degradation. Additional advantages include, under certain circumstances, increasing the stability and circulation time of the drug and decreasing immunogenicity.

The most commonly used water soluble polymer for conjugation to drugs is polyethylene glycol (“PEG”). The term “PEGylate” has come to mean the attachment of at least one PEG molecule to a second molecule. PEG molecules vary in size based on the number of repeated units in the structure and are typically described in molecular weights, for example, from a few hundred Daltons to 40-50 kiloDaltons or more.

PEGylation can extend the serum half life of a therapeutic protein thereby increasing the duration of its effectiveness and reducing the frequency of dosing. One mechanism by which PEG increases the serum half life of a protein based drug is by protecting against proteolysis, Sada, et al., J. Fermentation Bioengineering 71: 137-139 (1991). Indeed, chemical modification with a single 20 kDa polyethylene glycol (PEG) polymer at the N-terminus of leptin results in the need for less leptin to be administered and increases solubility relative to the unmodified native protein; see, e.g., PCT WO 96/40912. For a review, see Abuchowski et al., Enzymes as Drugs. (J. S. Holcerberg and J. Roberts, eds. pp. 367-383 (1981)).

Indeed, PEG is conjugated to several commercial therapeutic proteins and examples include PEG-adenosine deaminase (Adagen®) for treating severe combined immunodeficiency disease; Neulasta™ (pegfilgrastim) for treating neutropenia; Definity® Vial (Perflutren Lipid Microsphere) injectable suspension (PEG); Somavert® (pegvisomant) for treating acromegaly; PEG-L-asparaginase (Oncaspar®) for treatment of acute lymphoblastic leukemia or non-Hodgkin's lymphoma; and PEGASYS® (peginterferon alfa-2a), and PEG-INTRON® (peginterferon alfa-2b) for treating hepatitis.

However, there are limitations associated with such chemical modifications. For example, the use of these conjugates in chronic applications and/or in relatively large amounts can result in the accumulation of high molecular weight polymers due to their resistance to degradation. In addition, PEG-drug conjugates have been found to accumulate in kidney vacuoles when administered regularly over a period of time at high doses; see e.g., Conover et al., Artificial Organs, 21(5):369-378 (1997); Bendele et al., Toxicological Sciences, 42:152 (1998). Although it is not known if such vacuoles are detrimental to the health of an individual, it is preferable that drug administration has no associated abnormalities. Thus, it would be advantageous to have a water soluble polymer conjugated to a drug, where the water soluble polymer could be eliminated from a patient's body without unwanted accumulation, e.g., as measured by formation of kidney vacuoles.

### SUMMARY OF THE INVENTION

The present invention relates to water soluble block polymers linked by labile linkage groups, methods of making them and methods of using them. Accordingly, one aspect of the invention comprises a polymer covalently attached to another same (block homo-polymer) or different polymer (block co-polymer) through a linker and methods for using the same, wherein the polymer-linker structure can be repetitively attached to another polymer-linker to achieve a block polymer length as needed. In one embodiment, an A represents the polymer and a B represents the linker. Accordingly, the block homo-polymer has the structure of (A-B)ₙ, where n is an integer representing the desired number of repeat units.

In another embodiment, one polymer is represented by an A and the linker by a B and a second heterologous polymer is represented by C. In this block co-polymer example, the full polymer can interchangeably contain desired A-B or C-B units and these units can be alternated, e.g., (A-B-C-B-)ₙ where n is 1 to 1,000, or the block co-polymer may have each polymer type in variable numbers relative to each other within the polymer.

In another embodiment the labile linker is hydrolytically and/or proteolytically more sensitive than the internal molecular bonds of the water soluble polymer. Accordingly, the water soluble block polymer conjugated to a drug, when repeatedly and/or chronically administered, demonstrates increased serum half life and/or reduced antigenicity consistent with the advantages provided by conjugation to traditional water soluble polymers, for example after PEGylation, but diminishes or eliminates unwanted accumulation as measured by kidney vacuole formation.

As described below in more detail, the present invention has a number of aspects relating to chemically modifying drugs including proteins or analogs thereof. In certain aspects, the present invention relates to conjugation of a water soluble block polymer conjugated to a therapeutic protein. In particular aspects, the protein therapeutic is selected from leptin, a soluble tumor necrosis factor receptor (sTNFR), and a peptide designated L1-7.

As used herein, the water soluble block polymer is constructed from smaller polymer fragments of from 500 Daltons to up to 3,000 Daltons, with linkers between the polymer blocks that are hydrolytically or proteolytically sensitive to degradation. A representative polymer is polyethylene glycol (PEG) and a representative linkage is through an amide group.

### BRIEF DESCRIPTION OF THE FIGURES

FIG. 1. Kidney vacuole formation upon chronic injection of 1, 2, or 20 kDa PEGylated leptin is shown. On the Y-axis, 0 is no visible vacuoles, 0.5 is rare tiny vacuoles with sporadic distribution, 1.0 is minimal bead of tiny vacuoles under brushborder of cells in tubule, 1.5 is obvious small vacuoles, scattered, not affecting every cell or tubule, 2.0 is mild but obvious vacuoles in tubule with motheaten look, 2.5 is more severe than vacuoles but still not affecting entire cell volume, 3.0 is moderate, obvious, large, clear vacuoles in less than 50% of tubules, 3.5 is obvious vacuoles (as in 3.0) and nuclear degeneration, 4.0 is marked vacuolation of over 50% of tubules and nuclear degeneration.

FIG. 2. The weight loss of mice treated with twenty kiloDalton PEGylated leptin compared to leptin PEGylated with high, medium or low block polymers of the invention is depicted (square represent data from 20 kDa PEGylated-leptin, X represents data from low molecular weight block polymer-leptin conjugates, X with a strike through represents data from low molecular weight block polymer-leptin conjugates, + represents data from high molecular weight block polymer-leptin conjugates and the diamonds represent data from a PBS control.

FIG. 3. Kidney vacuole formation was measured in the mice treated according to FIG. 2.

FIG. 4. Data shows the results of a single injection of the conjugates made in Example 2. The key to the data is the same as FIG. 2, with the addition of triangles for Fc-leptin fusion proteins.

FIG. 5. Kidney vacuole formation was measured in the mice treated according to FIG. 3.

FIG. 6. Paw swelling and the effect of sTNFR1 conjugated either to a block polymer PEG or 20 kDa PEG is depicted.

### DETAILED DESCRIPTION

Conjugation of drugs, especially protein therapeutics, with water soluble polymers such as polyethylene glycol (PEG) confers important therapeutic benefits including reduced antigenicity and increased serum half life. However, chronic or high dosage administration of higher molecular weight polymer, e.g., over 5 kDa PEG, can result in delayed elimination from a subject as measured by accumulation of the polymers in, for example, kidney vacuoles.

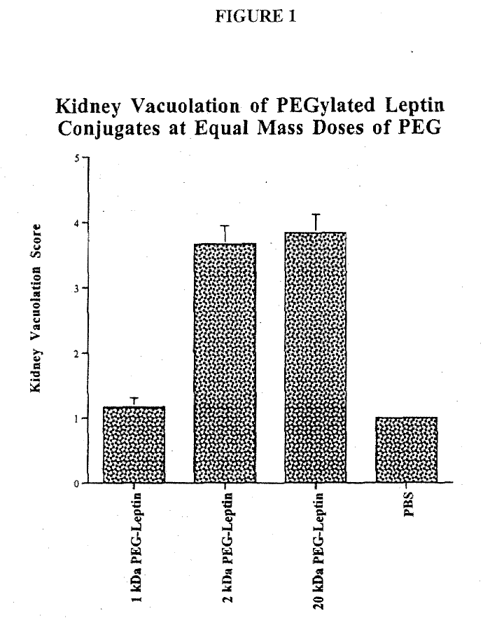

In contrast, it has been discovered that lower molecular weight forms of polymers, e.g., less than 2 kDa PEG, are cleared from serum without accumulating in kidney vacuoles. Indeed, there is a trend of increased vacuolization with increased molecular weight of PEG (FIG. 1). In these experiments, it was shown that when an equal mass of PEG of varying molecular weight, i.e., 1, 2, and 20 kDa, was mono-PEGylated onto a leptin molecule, only the 1 kDa PEG-leptin conjugate did not show measurable kidney vacuole formation in contrast to the 2 and 20 kDa PEG-leptin conjugates which did induce kidney vacuole formation. The molecular weight trend held for these poly PEGylated leptins as well and when leptin was multiply PEGylated with 1 kDa PEG, the kidney vacuole score was comparable to PBS controls or unmodified leptin. Furthermore, it was shown that the 1 kDa PEGylated leptin has equal activity to leptin in vivo, is more soluble at physiological pH, does not cause injection site reactions at high concentrations, and shows no evidence of kidney vacuolization. However, when a smaller PEG, e.g., less than 5 kDa, is conjugated to a drug, the increased serum half life is not provided as is found with larger PEG conjugates, e.g., 20 kDa PEG.

Accordingly, the present inventors have discovered that by linking blocks of water soluble molecules having around 1 kDa to either homologous or heterologous blocks via a labile linkage such that the total block polymer size is greater than 10 kDa the serum half life is increased. Importantly, this increase is comparable to conjugation to a non-block polymer and kidney vacuole accumulation found in chronic dosing of traditionally PEGylated proteins is reduced or eliminated.

Thus, the present invention relates to block polymers, methods of making and using these molecules, and block polymers conjugated to drugs. It is contemplated that the block polymers of the invention exhibit the pharmacological benefits of the large molecular weight polymers, e.g., increased serum half life and decreased immunogenicity, but are more degradable and accordingly don't have undesired properties. Working examples, described below, are provided of drugs conjugated to one example of block polymer of the invention and have similar pharmacokinetic properties to therapeutic molecules that are N-terminally mono-PEGylated with 20 kDa PEG. However, it is further demonstrated that the water soluble block polymer conjugated molecules are less prone to induce formation of kidney vacuoles upon chronic administration in contrast to those PEGylated with 20 kDa polymers.

The size of the block polymers is preferably about 10 to 50 kDa, more preferably 15 to 40 kDa and still more preferably 15 to 30 kDa, with a representative size being 20 kDa. It will be readily understood by one of ordinary skill in the art that different water soluble polymers will have some variability in properties and therefore the ideal size will need to be determined for use with the drug to be conjugated. The experimentation to determine the ideal composition and also the size of the blocks in the block polymer and the size of the block polymer are merely routine experiments in light of the disclosure herein.

As used herein, it is understood that a “labile linkage” is more susceptible to breakage either by a protease or hydrolytic degradation than the normal molecular bonds found in a water soluble polymer.

For the present block polymer compositions, one may select from a variety of water soluble polymers (by molecular weight, branching, etc.), the proportion of water soluble polymers to drug molecules in the reaction mix, the type of conjugation reaction to be performed, the method of obtaining the selected conjugated drug, and the type of drug to be used.

The block polymer should be water soluble so that the drug to which it is attached does not precipitate in an aqueous environment, such as a physiological environment. The polymer may be branched or unbranched. Preferably, for therapeutic use of the end-product preparation, the polymer will be pharmaceutically acceptable. One skilled in the art will be able to select the desired polymer based on such considerations as whether the polymer/protein conjugate will be used therapeutically, and if so, the desired dosage, circulation time, resistance to proteolysis, and other considerations.

Typical water soluble polymers suitable for conjugation to drugs include, but are not limited to, polyethylene glycols, copolymers of ethylene glycol/propylene glycol, carboxymethylcellulose, polyvinyl pyrrolidone, poly 1,3 dioxolane, poly 1,3,6 trioxane, ethylene/maleic anhydride copolymer, polyaminoacids, dextran or poly(n-vinyl pyrrolidone), propylene glycol homopolymers, propylene oxide/ethylene oxide polymers, polyoxyethylated polyols and polyvinyl alcohol. Several of these polymer-drug conjugate formulations have been shown to have improved pharmacological properties, e.g., enhanced serum half-life, improved stability, solubility and/or a reduction in immunogenicity has also been found (Trakas et al., J. Neuroimmunology, 120(1-2):42-9 (2001).

For water soluble polymers useful for the blocks in the block polymers of the present invention, the molecular weights are between about 500 Daltons and about 3000 Daltons. As used herein, the term “about” indicating that in preparations of water soluble polymers, some molecules will weigh more, some less, than the stated molecular weight, and the stated molecular weight is merely an average taken of the preparation. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). Accordingly, it is contemplated that polymers of about 500, about 600, about 700, about 800, about 900, about 1,000, about 1,100, about 1,200, about 1,300, about 1,400, about 1,500, about 1,600, about 1,700, about 1,800, about 1,900, or about 2,000 can be used according to the invention. The examples below involve the use of PEG 1000, which was selected for ease in purification and for providing an adequate model system.

In a particular embodiment, the water soluble polymer of the invention is a PEG based block polymer with utility as a carrier for drugs. In this example, the polymer can be synthesized by one step poly-addition polymerization of bifunctional low molecular weight PEG diamines (i.e., under 3,000 Daltons) and a bifunctional acid to yield a block polymer linked via amide groups. A thiol reactive polymer endgroup was formed by the reaction of maleimidopropionic acid NHS ester with an amino endgroup.

In one embodiment, the linker in the polymer of the invention is a diacid, for example, oxalylchloride. More particularly, in one example the block polymer-drug conjugate has the following formula: R′—[—HN—(CH₂CH₂O)ₙ—CH₂CH₂NH—C(═O)—C(═O)—]ₓ—NH—R—NHC(═O)CH₂CH₂-maleimide-S-drug, where R′ is a bifunctional group such as maleimide, R is a spacer molecule and can be a carbon, methylene or any other group that does not disrupt the structure and function of the molecule, n is 10 to 500 and x is 5 to 25.

In another example, the block polymer-labile linker has the formula R′—CH₂CH₂C(═O)NH—R—NH—C(═O)(CH₂)₃C(═O)—[—O(CH₂CH₂O)ₙ—CH₂CH₂O—C(═O)(CH₂)₃C(═O)—]ₓ—NH—R—NH—C(═O)CH₂CH₂-maleimide-drug, where the drug can be a protein or a peptide and where x is 10 to 1000 and R and R′ are linking groups. In another embodiment, the block polymer has the following formula: R—[—C(═O)—PEG1K—C(═O)—NH-PEG1K—NH—]ₙ—C(═O)—CH₂CH₂-maleimide-5-drug, where n is 3 to 30, more preferably 5 to 25, more preferably 7 to 20 and most preferably 10 to 15. The R groups in both cases can be any suitable capping group. In both of these examples the maleimide is reacted with a free thiol on the drug to form a thioether linkage with the drug. It is contemplated that the polymers of the invention are conjugated to a drug that is a protein that contains a cysteine residue to which the polymer is attached.

In the examples below, the PEG based block polymers were conjugated to a soluble tumor necrosis factor receptor (sTNFR), a peptide L1-7, and a leptin molecule containing a free cysteine to produce a block polymer-drug conjugate. However, it is understood that the polymers of the invention can be conjugated to any therapeutic molecule including proteins, peptides, (e.g., purified naturally occurring, recombinant, fusion, mutated, or synthetic proteins and/or peptides), or other molecules, so long as they have an appropriate linking group to conjugate with the polymer.

Representative examples of proteins and peptides useful herein include, but are not limited to, granulocyte colony stimulating factor (G-CSF), erythropoietin (EPO), antibodies, including IgG1, IgG2 and other isotypes, B1 antagonist peptides, insulin, gastrin, prolactin, adrenocorticotropic hormone (ACTH), thyroid stimulating hormone (TSH), luteinizing hormone (LH), follicle stimulating hormone (FSH), human chorionic gonadotropin (HCG), motilin, interferons (alpha, beta, gamma), interleukins (IL-1 to IL-12), tumor necrosis factor (TNF), tumor necrosis factor-binding protein (TNF-bp), brain derived neurotrophic factor (BDNF), glial derived neurotrophic factor (GDNF), neurotrophic factor 3 (NT3), fibroblast growth factors (FGF), neurotrophic growth factor (NGF), bone growth factors such as osteoprotegerin (OPG), insulin-like growth factors (IGFs), macrophage colony stimulating factor (M-CSF), granulocyte macrophage colony stimulating factor (GM-CSF), megakaryocyte derived growth factor (MGDF), keratinocyte growth factor (KGF), thrombopoietin, platelet-derived growth factor (PGDF), colony simulating growth factors (CSFs), bone morphogenetic protein (BMP), superoxide dismutase (SOD), tissue plasminogen activator (TPA), urokinase, streptokinase and kallikrein.

It is understood that methods applicable for the attachment of water soluble polymers such as PEG are also suitable for the attachment of the block polymers described herein. Thus, generally, water soluble molecules like polyethylene glycol are connected to drugs via a reactive group found on the drug. Amino groups, such as those on lysine residues or at the N-terminus of proteins, are convenient for such attachment. For example, Royer (U.S. Pat. No. 4,002,531, above) states that reductive alkylation was used for attachment of polyethylene glycol molecules to an enzyme. EP 0 539 167 states that peptides and organic compounds with free amino group(s) are modified with an intermediate derivative of PEG or related water-soluble organic polymers. U.S. Pat. No. 4,904,584, Shaw, issued Feb. 27, 1990, relates to the modification of the number of lysine residues in proteins for the attachment of polyethylene glycol molecules via reactive amine groups.

Additional methods for attachment of water soluble polymers are available. See, e.g., U.S. Pat. No. 4,179,337; and U.S. Pat. No. 4,002,531. U.S. Pat. No. 5,824,784, discloses N-terminally monopegylated proteins where “N-terminally monopegylated” denotes that the protein moiety has attached to it a single polyethylene glycol moiety at the N-terminus, and which demonstrate, inter alia, increased serum half-life and improved stability.

In yet another embodiment, the drug is conjugated to more than one polymer. Thus, various numbers of block polymer moieties may be attached to the drug (i.e., di-, tri-, tetra-, etc.). Likewise, a combination of polymer-drug conjugates may be combined into a mixture.

European patent publication EP 0 401 384 describes materials and methods for preparing G-CSF to which polyethylene glycol molecules are attached. A general method of N-terminal specific PEGylation of therapeutic proteins, including G-CSF and consensus interferon, is taught in U.S. Pat. No. 5,985,265. In addition, Pegylated IL-6 is taught in U.S. Pat. No. 5,264,209 which discloses polyethylene glycol molecules conjugated to IL-6. Further, international application No. WO/8503868 reports reacting a lymphokine with an aldehyde of polyethylene glycol.

In general, drugs useful in the practice of this invention may be a form isolated from chemical synthetic procedures or isolated from native mammalian organisms or, alternatively, from prokaryotic or eukaryotic host expression of exogenous DNA sequences obtained by genomic or cDNA cloning or by DNA synthesis. Suitable prokaryotic hosts include various bacteria (e.g., E. coli); suitable eukaryotic hosts include yeast (e.g., S. cerevisiae) and mammalian cells (e.g., Chinese hamster ovary cells, monkey cells). For example, proteins which are the product of an exogenous DNA sequence expressed in cells may have, as a result of expression, an N-terminal methionyl residue with an alpha-amino group. As indicated above, peptides are included, as are peptidomimetics and other modified proteins. Depending upon the host cell employed, a protein's expression product may be glycosylated with mammalian or other eukaryotic carbohydrates, or it may be non-glycosylated.

The protein expression product may also include an initial methionine amino acid residue (at position −1) and may be post-translational cleaved into a mature form, e.g., a secreted protein comprising a signal peptide may have the signal peptide cleaved. Protein analogs and the non-naturally occurring proteins, for example, consensus interferon, are also suitable for the present methods described in U.S. Pat. Nos. 5,824,784 and 5,985,265.

Generally, the usefulness of drugs and analogs thereof in the present invention may be ascertained by practicing the chemical modification procedures as provided herein to chemically modify the drug, and testing the resultant product for the desired characteristic, such as the biological activity assays. In the case of proteins, if one so desires when treating non-human mammals, one may use recombinant non-human protein's, such as recombinant murine, bovine, canine, etc. See PCT WO 9105798 and PCT WO 8910932, for example.

Further, the present compositions and methods include formulation of pharmaceutical compositions, methods of treatment and manufacture of medicaments.

The proportion of block polymers to drug molecules will vary, as will their concentrations in the reaction mixture. In general, the optimum ratio will be determined by the molecular weight of the polymer selected. In addition, one example involves non-specific pegylation and later purification of the desired polymer-drug species, the ratio may depend on the number of available amine reactive groups (typically amino groups) or free thiol groups that are available. One example of a low reaction ratio of drug to polymer molecules to obtain mono-polymer material is generally 1.5 polymer molecules per drug molecules. This ratio is particularly useful in protein to PEG conjugations.

A useful method of linking a water soluble polymer, e.g., a block polymer as described herein, to a protein involving no linking group between the polymer moiety and the protein moiety is described in Francis et al., (Eds. Ahern., T. and Manning, M. C.) Plenum, N.Y., 1991. Also, Delgado et al., Fisher et al., eds., Separations Using Aqueous Phase Systems, Applications In Cell Biology and Biotechnology, Plenum Press, N.Y. N.Y., 1989 pp. 211-213, involves the use of tresyl chloride, which results in no linkage group between a polyethylene glycol moiety and the protein moiety. This method may be difficult to use to produce therapeutic products as the use of tresyl chloride may produce toxic by-products. An alternative is the use of N-hydroxy succinimidyl esters of carboxymethyl methoxy polyethylene glycol.

It may be necessary to separate a particular species of block water soluble polymer linked drug, for example, isolation of an N-terminal conjugated protein, from other moieties if necessary. This purification involves separation from a population of conjugated proteins molecules. For example, one example is where conjugated protein is separated by ion exchange chromatography to obtain material having a charge characteristic of mono-conjugated material (other multi-conjugated material having the same apparent charge may be present), and then the mono-conjugated materials are separated using size exclusion chromatography. In this way, N-terminally conjugated protein can be separated from other mono-conjugated species, as well as other multi-conjugated species.

Other similar methods are reported. For example, PCT WO 90/04606, published May 3, 1990, teaches a process for fractionating a mixture of water soluble polymer-protein adducts comprising partitioning the conjugates in a polymer-containing aqueous biphasic system.

In another aspect, a water soluble polymer is conjugated to a protein selectively at the N-terminus. This includes modification by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved. The reaction is performed at pH, which allows one to take advantage of the pKₐ differences between the alpha-amino groups of the lysine residues and that of the alpha-amino group of the N-terminal residue of the protein. By such selective derivatization attachment of a water soluble polymer to a protein is controlled: the conjugation with the polymer takes place predominantly at the N-terminus of the protein and no significant modification of other reactive groups, such as the lysine side chain amino groups, occurs. According to this method, the monopolymer/protein conjugate will have a polymer moiety located at the N-terminus, but not on amino side groups, such as those for lysine. The preparation will preferably be greater than 80% monopolymer/protein conjugate, and more preferably greater than 95% monopolymer protein conjugate.

For reductive alkylation, the reducing agent should be stable in aqueous solution and preferably be able to reduce only the Schiff base formed in the initial process of reductive alkylation. Preferred reducing agents may be selected from the group consisting of sodium borohydride, sodium cyanoborohydride, dimethylamine borate, timethylamine borate and pyridine borate.

### Leptin Molecules

Leptin suitable for use in the present invention may be selected from the recombinant human and murine methionyl proteins. A particularly useful form of leptin is one where the native cysteines have been mutated and position 78 has been changed to a cysteine, leaving only the single cysteine as a reaction site for the maleimide group of the water soluble polymer. The native human and mouse leptin sequences are provided below.

Human Leptin:
MVPIQKVQDDTKTLIKTIVTRINDISHTQSVSAKQRVTGLDFIPGLHPIL
SLSKMDQTLAVYQQVLTSLPSQNVLQIANDLENLRDLLHLLAFSKSCSLP
QTSGLQKPESLDGVLEASLYSTEVVALSRLQGSLQDILQQLDVSPEC
Murine Leptin:
MVPIQKVQDDTKTLIKTIVTRINDISHTQSVSSKQRVTGLDFIPGLHPIL
TLSKMDQTLAVYQQILTSMPSRNVLQISNDLENLRDLLHVLAFSKSCHLP
WASGLETLDSLGGVLEASGYSTEVVALSRLQGSLQDMLWQLDLSPGC

Additionally, suitable leptins include those lacking a glutaminyl residue at position 28, where position one is contemplated to be the first valine and the first methionine is position −1 (Zhang et al., Nature 372: 425-432 (1994); see also, the Correction at Nature 374: 479 (1995)). The recombinant human leptin gene product is, as a mature protein, 146 amino acids and lacks an N-terminal methionine. The murine protein is substantially homologous to the human protein, particularly as a mature protein, and, further, particularly at the N-terminus. One may prepare an analog of the recombinant human protein by altering (such as substituting amino acid residues), in the recombinant human sequence, the amino acids which diverge from the murine sequence. Because the recombinant human protein has biological activity in mice, such analog would likely be active. Proteins lacking an N-terminal methionyl residue, such as those produced by eukaryotic expression are also available for use.

### Soluble Tumor Necrosis Factor Binding Proteins

For purposes of this invention, the molecules disclosed in the following references relating to TNF inhibitors and the sTNFRs and variants and derivatives of the sTNFRs and the molecules disclosed in the references (see below) are collectively termed “TNF-alpha inhibitors.” These include soluble forms of the TNF receptor type I or type II. As used in the examples below, it is noted that a TNF binding protein is used, however, it is contemplated that additional polypeptides are useful with the compositions and in the methods described herein. Representative TNF binding proteins are described in U.S. Pat. Nos. 6,541,620, 6,271,346, and 6,143,866.

U.S. Pat. No. 6,541,620 teaches the sequences of soluble TNT receptor type I (also known as sTNFR-I or 30 kDa TNF inhibitor) and soluble TNF receptor type II (also known as sTNFR-II or 40 kDa TNF inhibitor), collectively termed “sTNFRs”, as well as modified forms thereof (e.g., fragments, functional derivatives and variants). Further, EP 393 438 teaches a 40 kDa TNF inhibitor D51 and a 40 kDa TNF inhibitor D53, which are truncated versions of the full-length recombinant 40 kDa TNF inhibitor protein wherein 51 or 53 amino acid residues, respectively, at the carboxyl terminus of the mature protein are removed. sTNFR-I and sTNFR-II are members of the nerve growth factor/TNF receptor superfamily of receptors which includes the nerve growth factor receptor (NGF), the B cell antigen CD40, 4-1BB, the T-cell antigen OX40, the Fas antigen, and the CD27 and CD30 antigens (Smith et al. (1990), Science, 248:1019-1023).

PCT Application No. PCT/US97/12244 teaches truncated forms of sTNFR-I and sTNFR-II which do not contain the fourth domain (amino acid residues Thr127-Asn161 of sTNFR-I and amino acid residues Pro141-Thr179 of sTNFR-II); a portion of the third domain (amino acid residues Asn111-Cys126 of sTNFR-I and amino acid residues Pro123-Lys140 of sTNFR-II); and, optionally, which do not contain a portion of the first domain (amino acid residues Asp1-Cys19 of sTNFR-I and amino acid residues Leu1-Cys32 of sTTR-II). The truncated sTNFRs useful in the present invention include the proteins represented by the formula R1-[Cys19-Cys103]-R2 and R4-[Cys32-Cys115]-R5. These proteins are truncated forms of sTNFR-I and sTNFR-II, respectively, and provide opportunity for dual modification at the thiol side groups provided by the cysteine amino acids. By “R1-[Cys19-Cys103]-R2” is meant one or more proteins wherein [Cys19-Cys103] represents residues 19 through 103 of sTNFR-I, the amino acid residue; wherein R1 represents a methionylated or nonmethionylated amine group of Cys19 or of amino-terminus amino acid residue(s) selected from any one of Cys18 to Asp1 and wherein R2 represents a carboxy group of Cys103 or of carboxy-terminal amino acid residues selected from any one of Phe104 to Leu110.

Exemplary truncated sTNFR-I of the present invention include the following molecules (collectively termed 2.6D sTNFR-I): NH2-[Asp1-Cys105]-COOH (also referred to as sTNFR-I 2.6D/C105); NH2-[Asp1-Leu108]-COOH (also referred to as sTNFR-I 2.6D/C106); NH2-[Asp1-Asn105]-COOH (also referred to as sTNFR-I 2.6D/N105); NH2-[Tyr9-Leu108]-COOH (also referred to as sTNFR-I 2.3D/d8); NH2-[Cys19-Leu108]-COOH (also referred to as sTNFR-I 2.3D/d18); and NH2-[Ser16-Leu108]-COOH (also referred to as sTNFR-I 2.3D/d15), either methionylated or nonmethionylated, and variants and derivatives thereof.

TNF-alpha inhibitors of various kinds are disclosed in the art, including the following references: U.S. Pat. Nos. 5,136,021; 5,929,117; 5,948,638; 5,807,862; 5,695,953; 5,834,435; 5,817,822; 5,830,742; 5,834,435; 5,851,556; 5,853,977; 5,359,037; 5,512,544; 5,695,953; 5,811,261; 5,633,145; 5,863,926; 5,866,616; 5,641,673; 5,869,677; 5,869,511; 5,872,146; 5,854,003; 5,856,161; 5,877,222; 5,877,200; 5,877,151; 5,886,010; 5,869,660; 5,859,207; 5,891,883; 5,877,180; 5,955,480; 5,955,476; 5,955,435; 5,994,351; 5,990,119; 5,952,320; 5,962,481. The relevant portions of these disclosures are incorporated herein by reference.

### Methods of Treatment

In yet another aspect of the present invention, methods of treatment and manufacture of a medicament are provided. Conditions alleviated or modulated by the administration of the present block polymer-drug conjugate depend on the drug which is conjugated. For example, when the drug is leptin, conditions may be alleviated or modulated by administration of the present block polymer-leptin conjugates are those to which leptin is applicable and include obesity. The working examples below demonstrates that leptin chemically modified with a block polymer of the invention is approximately as active as a leptin chemically modified with a PEG molecule. Likewise, a sTNFR conjugated to a block polymer of the invention is shown to be effective in treating an inflammatory condition, while also being less prone to inducing kidney vacuole formation.

A soluble tumor necrosis factor receptor conjugated to a block polymer of the invention can be used to treat conditions associated over expression of TNF, for example inflammation. A non-exclusive list of acute and chronic TNF-mediated diseases that can be treated with TNF inhibitor compositions of the invention includes, but is not limited to the following: cachexia/anorexia; cancer (e.g., leukemia's); chronic fatigue syndrome; coronary conditions and indications, including congestive heart failure, coronary restenosis, myocardial infarction, myocardial dysfunction (e.g., related to sepsis), and coronary artery bypass graft; depression; diabetes, including juvenile onset Type 1, diabetes mellitus, and insulin resistance (e.g., as associated with obesity); endometriosis, endometritis, and related conditions; fibromyalgia or analgesia; graft versus host rejection; hyperalgesia; inflammatory bowel diseases, including Crohn's disease and Clostridium difficile-associated diarrhea; ischemia, including cerebral ischemia (brain injury as a result of trauma, epilepsy, hemorrhage or stroke, each of which may lead to neurodegeneration); lung diseases (e.g., adult respiratory distress syndrome, asthma, and pulmonary fibrosis); multiple sclerosis; neuro-inflammatory diseases; ocular diseases and conditions, including corneal transplant, ocular degeneration and uveitis; pain, including cancer-related pain; pancreatitis; periodontal diseases; Pityriasis rubra pilaris (PRP); prostatitis (bacterial or non-bacterial) and related conditions; psoriasis and related conditions; pulmonary fibrosis; reperfusion injury; rheumatic diseases, including rheumatoid arthritis, osteoarthritis, juvenile (rheumatoid) arthritis, seronegative polyarthritis, ankylosing spondylitis, Reiter's syndrome and reactive arthritis, Still's disease, psoriatic arthritis, enteropathic arthritis, polymyositis, dermatomyositis, scleroderma, systemic sclerosis, vasculitis (e.g., Kawasaki's disease), cerebral vasculitis, Lyme disease, staphylococcal-induced (“septic”) arthritis, Sjögren's syndrome, rheumatic fever, polychondritis and polymyalgia rheumatica and giant cell arteritis); septic shock; side effects from radiation therapy; systemic lupus erythematosus (SLE); temporal mandibular joint disease; thyroiditis; tissue transplantation or an inflammatory condition resulting from strain, sprain, cartilage damage, trauma, orthopedic surgery, infection (e.g., HIV, Clostridium difficile and related species) or other disease process.

Another aspect described herein is pharmaceutical compositions of the above. Such pharmaceutical compositions may be for administration for injection, or for oral, pulmonary, nasal or other forms of administration. In general, comprehended by the invention are pharmaceutical compositions comprising effective amounts of monopolymer/protein conjugate products together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, or into liposomes. Such compositions may influence the physical state, stability, rate of in vivo release, and rate of in vivo clearance of the present chemically modified proteins. See, e.g., Remington's Pharmaceutical Sciences, 18th Ed. (1990, Mack Publishing Co., Easton, Pa. 18042) pages 1435-1712 which are herein incorporated by reference.

As further studies are conducted, information will emerge regarding appropriate dosage levels for treatment of various conditions in various patients, and the ordinary skilled worker, considering the therapeutic context, age and general health of the recipient, will be able to ascertain proper dosing. Generally, for injection or infusion, dosage will be between 0.01 μg/kg body weight, (calculating the mass of the protein alone, without chemical modification), and 100 μg/kg (based on the same).

The below examples illustrate the various aspects discussed above.

### EXAMPLE 1

### (PEG1K+GLUT-Polyester)-Leptin

### Materials

A polyethylene glycol with two hydroxy end groups (PEG1K-diol) of M_{w}=1000 KDa (Aldrich) was dried in a vacuum oven (50-60° C.) for overnight prior to use. Glutaryl chloride (Fluka), anhydrous chloroform (Aldrich), anhydrous diether (J T Baker), anhydrous isopropanol (Aldrich), 2,2′-(ethylenedioxy)bis(ethylamine) (Fluka), triethylamine (99%+, B&J), N,N-dicyclohexylcarbodiimide (DCC, Lancaster) and sterile water (Baxter) were used as received.

### Instrumentation

Pall Filtrons (3 kDa nominal molecular weight cut-off membranes) and Slide-A-Lyzer cassettes (Pierce, 7 kDa nominal molecular weight cut-off) were used for concentration, purification or buffer exchange. GPC was carried out using poly(ethylene glycol) standards with Millenium32 software using a Waters System 717 Autosampler, 510 Pump, 490E multiwavelength detector and a 410 differential refractometer; Alpha Series (TSK) 2500 and 4000 columns with a mobile phase of 20 mM LiBr in methanol. FPLC (Pharmacia) was carried out on a HiLoad SP 26/10 cation exchange column eluting at 2.5 ml/min with 20 mM sodium acetate pH 4.0 with a 0-55% gradient of 20 mM sodium acetate pH 4.0 plus 0.5 M NaCl. Thin layer chromatography (TLC) was carried out on 100 plates from EM Science (TLC 60° F₂₅₄) developed with methanol/methylene chloride (1:4) and visualized with iodine vapor and ninhydrin spray.

### Polymerization

PEG1K-diol (24.06 g, 24.06 mmol) was dissolved in anhydrous chloroform (60 ml) and triethylamine (4.87 g, 48.12 mmol) added. The reaction mixture was stirred in an ice bath and glutaryl chloride (4.07 g, 24.08 mol) in 5 ml of anhydrous chloroform was added dropwise during 5-6 hours. The reaction was stirred at room temperature for overnight. The product molecular weight was followed by GPC until the desired range (20-50 KDa) was achieved at which point additional 0.5 M glutaryl chloride (2.03 g, 12.01 mmol) was added and the reaction was left at room temperature for overnight. To end-cap the polymer with amines, a five-fold excess of 2,2′-(ethylenedioxy)bis(ethylamine) (17.83 g, 120 mmol) in 200 ml of anhydrous chloroform was added dropwise into the reaction mixture. The capping reaction was monitored until the TLC ninhydrin positive polymer spot showed no further change. The polymer was precipitated with a cold (4° C.) solvent mixture of chloroform: ether: isopropanol (400 ml: 1333 ml: 667 ml). The precipitate was filtered and dried under reduced pressure.

### Activation

A solution of maleimidopropionic acid (Sigma, 0.738 g, 4.36 mmol) and triethylamine (3.05 g, 30.1 mmol) in 100 ml of dichloromethane was treated with DCC (18.77 mg, 90.96 mmol) for 1 hr, then the above amine-capped polymer (3.055 g, 152.8 μmol, Mₙ=20 K, polydispersity=1.3-1.5) was added. The reaction was left at 4° C. for 2 days. The precipitated dicyclohexylurea was removed by filtration. The filtrate was diluted with a mixture of chloroform: ether: isopropanol (120 ml: 800 ml: 400 ml) and the precipitate removed by filtration. The filtrate was dried under reduced pressure yielding 1.863 g (61.0%) of crude product that was then dialyzed against 2 L of 20 mM phosphate, 5 mM EDTA, and pH 6.5 in Slide-A-Lyzer cassette.

### Conjugation

Polymer (1.8269 g, 90 μmol) solution in 20 mM phosphate buffer, pH 6.5 containing 5 mM EDTA and Leptin S78C (97 mg, 6.0 μmol) was added to give a final protein concentration of 1 mg/ml. The reaction was incubated at 4° C. for overnight.

### Purification

The polymer conjugate reaction mixture pH was adjusted to 3.5 then purified by FPLC using a HiLoad SP 26/10 column. Fractions 18-27 were combined as the high MW conjugate, fractions 28-30 were combined as medium MW conjugate and fractions 31-32 were combined as the low MW conjugate.

To prepare the samples for bioassay, the high, medium and low MW pools were concentrated in Pall Filtron centrifugal concentrators (MWCO 3.5 kDa) and then each pool was dialyzed against PBS twice (1 L) at 4° C. over a period of 24 hrs using 3.5 kDa MWCO membrane. Finally, the samples were concentrated to 2 mg/ml and filtered through Acrodic Syringe filters (25 mm, 0.2 μm HT Tuffryn membrane, Gelman Laboratory) into 5 ml sterile vials.

### Results

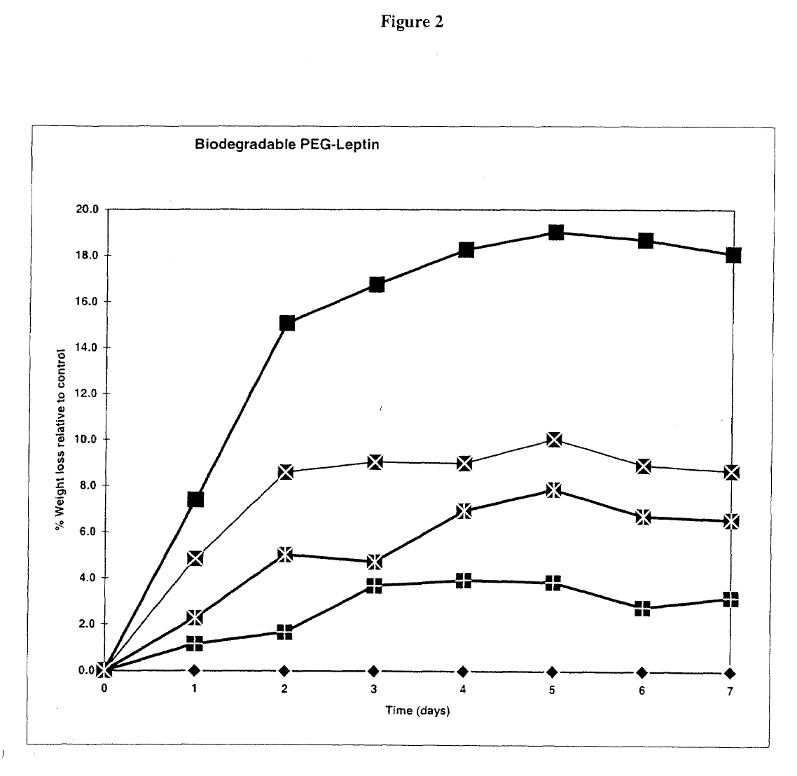

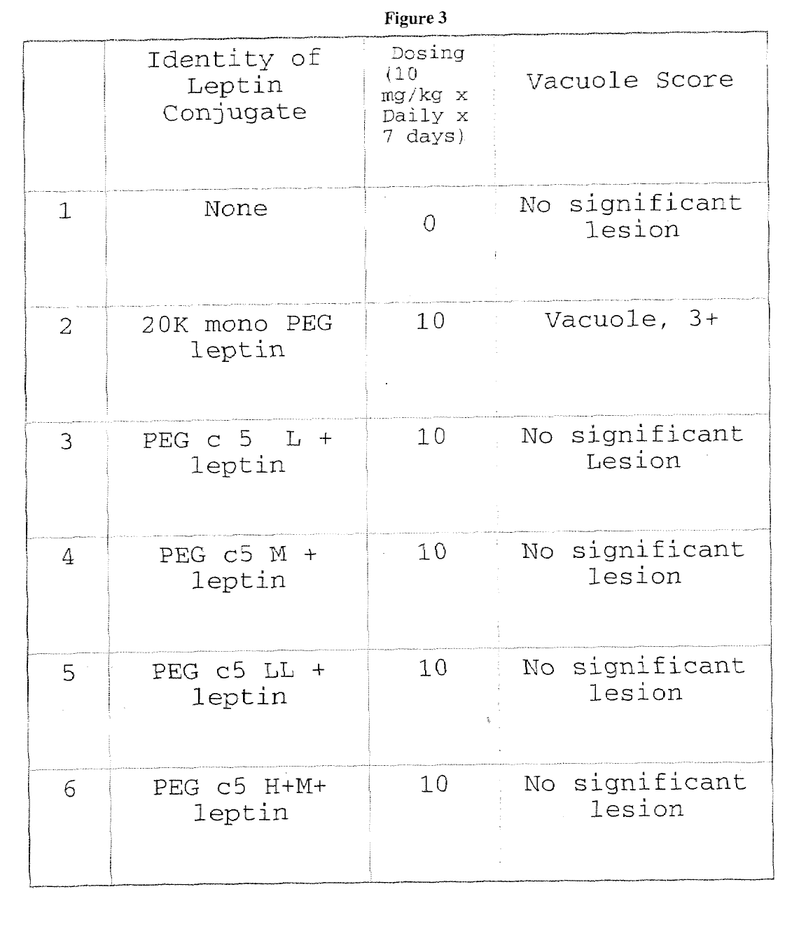

PEG1K-GLUT-Polyester-Leptin was tested for in vitro resistance to hydrolysis and efficacy in inducing weight loss in animals as shown in FIGS. 1 and 2. The data shows that mice treated with PEGylated leptin and the leptin PEGylated with the block polymers of the invention lose weight (FIG. 2) and the formation of kidney vacuoles in the block polymer-leptin conjugates is substantially reduced such that the kidney vacuoles were undetectable (FIG. 3).

### EXAMPLE 2

### PEG1K+OXL-Leptin

### Materials

A polyethylene glycol with two amine end groups (PEG1K-diamine) of M_{w}=1 KDa (Shearwater Polymer Inc.) was dried in a vacuum oven at 50° C.-60° C. for overnight, then cooled to room temperature. Oxalyl chloride (99+%, Fluka) dichloromethane (HPLC grade, Mallinckrodt), anhydrous diethylether (J T Baker), methanol (HPLC grade, J T Baker), anhydrous acetonitrile (Aldrich), 2,2′-(ethylenedioxy)bis(ethylamine) (Fluka), triethylamine (>99.9%, Romil Ltd.) and sterile water (Baxter) were used as received.

### Instrumentation

Amicon Stirred Cells with YM-3 and YM-10 membranes (3000 and 10,000 KDa nominal molecular weight cut-offs) were used for purification. GPC was carried out using poly(ethylene glycol) standards with Millenium32 software using a Waters System 717 Autosampler, 510 Pump, 490E multiwavelength detector and a 410 differential refractometer; Alpha Series 2500 and 4000 columns (TSK) with a mobile phase of 20 mM LiBr in methanol. FPLC (Pharmacia) was carried out on a HiLoad SP 26/10 cation exchange column eluting at 2.5 ml/mm with 20 mM sodium acetate pH 4.0 with a 0-55% gradient at 25 CVs, 20 mM sodium acetate pH 4.0 plus 0.5 M NaCl. Thin layer chromatography (TLC) was carried out on 100 plates from EM Science (TLC 60° F₂₅₄) developed with methanol/chloroform (1:4) and visualized with iodine vapor and ninhydrin spray.

### Polymerization

PEG1K-diamine (7.10 g, 7.10 mmol) was dissolved in anhydrous acetonitrile (110 ml) and triethylamine (1.44 g, 14.20 mmol) was added. The reaction mixture was stirred in a dry ice bath and oxalyl chloride (0.9 g, 7.10 mmol) in 10 ml of anhydrous acetonitrile was added dropwise during 3-4 hours, and then stirred at room temperature for overnight. The product molecular weight was followed by GPC until the desired range (20-50 kDa) was achieved at which point 2,2′-(ethylenedioxy)bis(ethylamine) (1.05 g, 0.2 mmol) in 70 ml of anhydrous acetonitrile was added. The capping reaction was followed until the TLC ninhydrin positive polymer spot showed no further change. The acetonitrile was evaporated under reduced pressure with a rotary evaporator leaving an oily residue. The oily residue was dissolved in 20 ml of sterile water and purified with 2 L of sterile water in a stir cell employing a YM-10 membrane. The bulk of the water was removed by evaporation and the remainder removed by azeotropic distillation (three times 100 ml toluene) with a rotary evaporator. The residue was precipitated with 200 ml of anhydrous diethylether and dried under vacuum. The weight of the product was 5.18 g. (64.7% by weight).

### Activation

The above amine capped polymer (5.18 g, 287 μmol, Mₙ=18 K, polydispersity=1.35) was dissolved in anhydrous acetonitrile (30 ml) and treated with triethylamine (48.2 mg, 476 μmol) and maleimidopropionic acid NHS ester (Bioscience, 0.2287 g, 859 μmol). The activation reaction was followed until the TLC ninhydrin positive polymer spot became ninhydrin negative. The reaction was found to be complete after 1 hour. The solvent was evaporated and the product purified with 1 L of sterile water in a stirred cell employing a YM-10 membrane The bulk of the water was removed by evaporation and the remainder removed azeotropically (3×30 ml toluene) then vacuum dried for overnight. The product weighted 3.59 g (˜69% by weight).

### Conjugation

Polymer (610 mg, 33.8 μmol) was dissolved in 20 mM phosphate, 5 mM EDTA pH 6.5. Leptin S78C (100 mg, 6.2 μmol) was added. The reaction was incubated at 4° C. overnight.

### Purification

The polymer conjugate solution was adjusted to pH 3.5 prior to loading onto the column. The conjugate was purified by FPLC employing 20 mM sodium acetate pH4 and 20 mM sodium acetate plus 0.5 mM NaCl as eluants. Fractions 32-50 were combined as the high MW conjugate, fractions 51-60 were combined as the medium MW conjugate and fractions 61-68 were combined as the low MW conjugate.

To prepare the sample for bioassay, the high, medium and low MW pools were concentrated in Amicon stirred cells (YM-3 membrane) and then each pool was dialyzed against PBS twice (1 L) at 4° C. over a period 24 hrs using 3.5 kDa MWCO membrane. Finally, the samples were concentrated to 2 mg/ml and filtered (25 mm, 0.2 um Acrodicc syringe filters, HT Tuffryn membrane, Gelman laboratory) into 5 ml sterile vials.

### Results

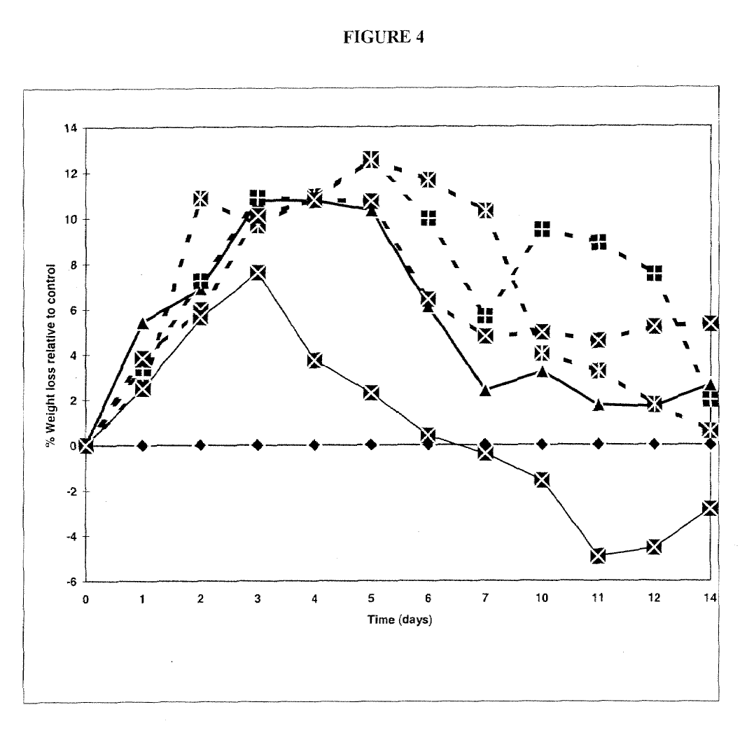

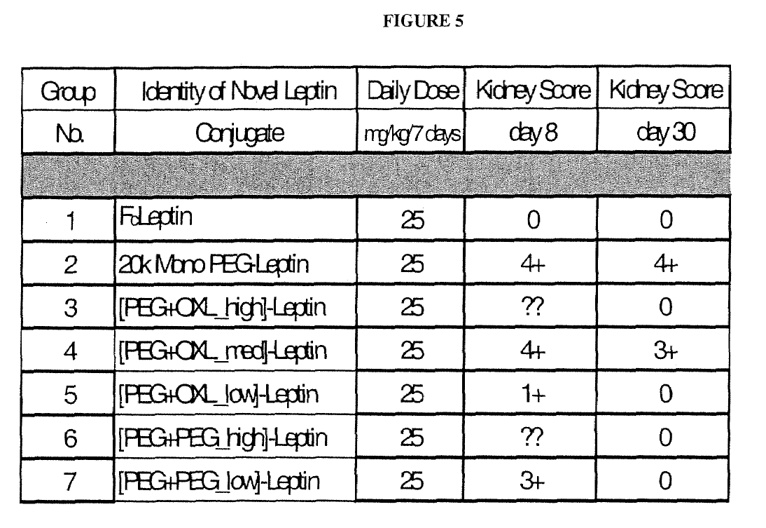

Mice were injected with a single dose of low, medium and high molecular weight PEG block polymers conjugated to leptin (described in this example, above), Fc-leptin fusion protein or 20 kDa PEGylated leptin. The results of the weight loss from these injections are depicted in FIG. 4 and the kidney vacuole formation is tabulated in FIG. 5. These results clearly demonstrate an improvement of the block polymers conjugated to a protein compared to regular PEG polymers conjugated to the same protein.

### EXAMPLE 3

### PEG1K+PEG1K-Leptin

### Materials

A polyethylene glycol with two amine end groups (PEG1K-diamine) of M_{w}=1000 KDa (Shearwater Polymer Inc.) and a polyethylene glycol with two carboxylic acid end groups (PEG1K-diacid) of M_{w}=1056 KDa (Shearwater Polymer Inc.) were each azeotropically dried on a rotary evaporator (50° C.-60° C., 3×, 30 ml toluene). Dichloromethane (HPLC grade, Mallinckrodt), thionyl chloride (99% low iron, Aldrich), N,N-dimethyformamide (99.8%, Aldrich), methanol (HPLC grade, J T Baker), anhydrous acetonitrile (Aldrich), 2,2′-(ethylenedioxy)bis(ethylamine) (Fluka), triethylamine, (99.9%, Romil Ltd.) and sterile water (Baxter) were used as received.

### Instrumentation

Amicon Stirred Cells with YM-3 and YM-10 membranes (3000 and 10,000 kDa nominal molecular weight cut-offs) were used for purification. GPC was carried out using poly(ethylene glycol) standards with Millenium32 software using a Waters System 717 Autosampler, 510 Pump, 490E multiwavelength detector and a 410 differential refractometer; Alpha Series 2500 and 4000 columns with a mobile phase of 20 mM LiBr and 0.1% triethylamine in methanol. FPLC (Pharmacia) was carried out on a HiLoad SP 26/10 cation exchange column eluting at 2.5 ml/min with 20 mM sodium acetate pH 4.0 with a 0-55% gradient of 20 mM sodium acetate pH 4.0 plus 0.5 M NaCl. Thin layer chromatography (TLC) was carried out on 100 plates from EM Science (TLC 60° F₂₅₄) developed with methanol/chloroform (1:4) and visualized with iodine vapor and ninhydrin spray.

### PEG1K-Diacid Chloride

Distilled thionyl chloride (2.13 g, 17.91 mmol) and DMF (0.142 g, 1.94 mmol) in 35 ml toluene were slowly introduced into a 100 ml round bottom flask containing PEG1K-diacid (4.7289 g, 4.478 mmol). The reaction was allowed to react at room temperature for an additional 1-2 hours and then concentrated on a rotary evaporator (50-60° C.) leaving an oily residue of PEG1K-diacid chloride, which was stored under argon gas.

### Polymerization

PEG1K-diamine (4.52 g, 4.52 mmol) was dissolved in anhydrous acetonitrile (50 ml) and triethylamine (0.9145 g, 9.04 mmol) added. The reaction mixture was stirred at room temperature and PEG1K-diacid chloride (4.7289 g, 4.478 mmol) in 30 ml anhydrous acetonitrile was added dropwise during 3-4 hours, then the reaction was stirred at room temperature for overnight. The product molecular weight was followed by GPC until the desired range (20-50 KDa) was achieved at which point 2,2′-(ethylenedioxy)bis(ethylamine) (0.034 g, 0.23 mmol) was added. The capping reaction was monitored until the TLC ninhydrin positive polymer spot showed no further change. The acetonitrile was evaporated under reduced pressure on a rotary evaporator leaving an oily residue. The oily residue was dissolved in 20 ml sterile water and purified with 2 L of sterile water in a stirred cell employing a YM-10 membrane. The solution was concentrated and residual water removed by azeotropic (three times 100 ml toluene) distillation on a rotor evaporator. The weight of the product was 3.7 g. (40% yield).

### Activation

The above amine capped polymer (3.76 g, 188 μmol, Mₙ=20 kDa, polydispersity=1.64) was dissolved in anhydrous acetonitrile (40 ml) and treated with triethylamine (28.55 mg, 282 μmol) and maleimidopropionic acid NHS ester (Bioscience, 76.8 mg, 288 μmol). The reaction was found to be complete by TLC (ninhydrin negative) after 1 hour. The solvent was evaporated and the product purified with 1 L of sterile water in a stirred cell employing a YM-3 membrane. The solution was concentrated, azeotropically dried (three times, 30 ml toluene) and vacuum dried for overnight. The product weighed 3.13 g (84.7% yield).

### Conjugation

The activated maleimide polymer (3.13 g) was dissolved in 50 ml of 20 mM Phosphate, 5 mM EDTA, and pH 6.5 buffer. Leptin S78C (126 mg, 7.798 μmol) was added. The final concentration of the reaction was 2 mg/ml of the protein (total 65 ml buffer). The reaction mixture was left at 4° C. for overnight.

### Purification

The polymer conjugate pH was adjusted to 3.5 and diluted to 1 mg protein/ml. The conjugation was purified by FPLC using 20 mM sodium acetate pH4 and 20 mM sodium acetate plus 0.5 M NaCl as eluants. Fractions were analyzed on 4-20% tri-gly mini gels (Novex, Coomassie Blue staining). Fractions 29-49 were combined as the high MW conjugate, fractions 50-57 were combined as medium MW conjugate and fractions 58-65 were combined as the low MW conjugate.

To prepare the samples for bioassay the high, med, and low MW pools were concentrated and the buffer exchanged to PBS in an Amicon stirred cell (YM-3 membrane) at 4° C. to a concentration of 5 mg/ml. The samples were diluted to 2 mg/ml and 0.2 mg/ml and filtered through Acrodic Syringe filters (25 mm, 0.2 μm HT Tuffryn membrane, Gelman Laboratory) into 5 ml sterile vials.

### EXAMPLES 1-3 CONCLUSIONS

The amide-leptin polymers described above were formulated and injected into mice and weight loss was measured over a controlled period. Mice injected with PBS were used as a control and their weights were used to calculate a baseline of zero. An Fc-leptin fusion at 10 mg/kg in a single dose at day zero induced a peak of 11% weight loss at day four with the weight returning to near the baseline by day eight. Likewise, the PEG1k+oxl high molecular weight conjugated, medium molecular weight conjugated and low molecular weight conjugated leptin conjugates, also at 10 mg/kg in single doses, induced a peak weight loss at around days four or five. However, in contrast to Fc-leptin, the PEG1k+oxl medium molecular weight conjugated leptin and PEG1k+oxl high molecular weight conjugated leptin conjugates maintained the weight loss beyond the test period of fourteen days above 2%.

A 20 k mono PEG-leptin conjugate was also administered at 10 mg/kg in a single dose and induced peak weight loss at day three at 9%, and the animals gained weight to return within 1% of baseline by day eight, and was below baseline weight before day 11 (FIG. 4). These experiments were repeated with comparable results.

In a separate experiment, kidney vacuoles were identified after injecting the conjugates and were scored using the following scale. Grade zero equals no kidney vacuoles. Grade 1+ equals minimal kidney vacuoles represented by rare, small vacuoles. Grade 2+ equals mild kidney vacuoles represented by modest numbers of about 3 micrometer diameter vacuoles. Grade 3+ equals moderate kidney vacuoles represented by large numbers of about 3 to about 5 micrometer diameter vacuoles. Grade 4+ equals marked kidney vacuoles meaning there are myriad and large, i.e., over 5 micrometer in diameter vacuoles. The renal tubular epithelium was the primary site analyzed for vacuole formation.

C57BL/6 mice were injected with a daily dose of 10 or 25 mg/kg conjugate or control for either seven or fourteen days. PEG1k+oxl high molecular weight conjugated leptin did not induce vacuoles in renal tubular epithelium after daily subcutaneous injection at either seven or fourteen days.

PEG1k+oxl medium molecular weight conjugated leptin induced renal epithelial vacuoles after daily injections at 25 mg/kg for seven days at grade 1+ or fourteen days at grade 2+ and induced minimal kidney vacuoles (grade 1+) after 10 mg/kg were injected daily for fourteen days. There were no detected kidney vacuoles when 10 mg/kg was injected daily for seven days with this conjugate.

PEG1k+oxl low molecular weight conjugated leptin induced kidney vacuoles after daily subcutaneous injections at 25 mg/kg for seven days (grade 2+) or fourteen days (grade 3+). Lesions were also observed after administration of 10 mg/kg/day for seven days (grade 2+) or fourteen days (grade 2+).

The positive control, 20 k mono PEG leptin induced kidney vacuoles after injection of 10 mg/kg/day or 25 mg/kg/day with a minimum of grade 3+ kidney vacuoles regardless of the dosing period.

### EXAMPLE 4

### PEG1K+OXL/L1-7 Peptide

PEG1K+OXL polymer (MW=15 KDa, 96.29 mg (6.4 micromoles) was added to a solution of L1-7 peptide (5.39 mg, 1.5 micromoles) in 50 mM Phosphate, 5 mM EDTA pH 6.5. The final concentration of the peptide was 2.5 mg/ml. The solution was incubated at 4° C. for overnight. Separation of the conjugate into different molecular weight pools was carried out via FPLC using a Hi-Trap SP HP 1 ml column. The column was equilibrated with 5 CV of 20 mM sodium acetate pH 4.0 and the conjugate was eluted into 1 ml fraction with 30 CV of 20 mM sodium Acetate plus 500 mM sodium Chloride pH 4.0. A high MW pool was formed from fractions 1-4 and a low MW pool from fractions 9-39.

Both pools were concentrated and buffer exchanged to PBS.

### EXAMPLE 5

### PEG 1K+OXL-sTNF

### Materials

A polyethylene glycol with two amine end groups (PEG1K-diamine) of M_{w}=1 KDa (Shearwater Polymer Inc.) was dried in a vacuum oven at 50° C.-60° C. for overnight, then cooled to room temperature. Oxalylchloride (99+%, Fluka) dichloromethane (HPLC grade, Mallinckrodt), anhydrous diethylether (J T Baker), methanol (HPLC grade, J T Baker), anhydrous acetonitrile (Aldrich), 2,2′-(ethylenedioxy)bis(ethylamine) (Fluka), triethylamine (>99.9%, Romil Ltd.) and sterile water (Baxter) were used as received.

### Instrumentation

Amicon Stirred Cells with YM-3 and YM-10 membranes (3000 and 10,000 KDa nominal molecular weight cut-offs) were used for purification. GPC was carried out using poly(ethylene glycol) standards with Millenium32 software using a Waters System 717 Autosampler, 510 Pump, 490E multi-wavelength detector and a 410 differential refractometer; Alpha Series 2500 and 4000 columns (TSK) with a mobile phase of 20 mM LiBr in methanol. FPLC (Pharmacia) was carried out on a HiLoad SP 26/10 cation exchange column eluting at 2.5 ml/min with 20 mM sodium acetate pH 4.0 with a 0-55% gradient of 20 mM sodium acetate pH 4.0 plus 0.5 M NaCl. Thin layer chromatography (TLC) was carried out on 100 plates from EM Science (TLC 60° F₂₅₄) developed with methanol/chloroform (1:4) and visualized with iodine vapor and ninhydrin spray.

### Polymerization

PEG1K-diamine (7.10 g, 7.10 mmol) was dissolved in anhydrous acetonitrile (110 ml) and triethylamine (1.44 g, 14.20 mmol) was added. The reaction mixture was stirred in a dry ice bath and oxalylchloride (0.9 g, 7.10 mmol) in 10 ml of anhydrous acetonitrile was added dropwise during 3-4 hours, and then stirred at room temperature for overnight. The product molecular weight was followed by GPC until the desired range (20-50 kDa) was achieved at which point 2,2′-(ethylenedioxy)bis(ethylamine) (1.05 g, 0.2 mmol) in 70 ml of anhydrous acetonitrile was added. The capping reaction was followed until the TLC ninhydrin positive polymer spot showed no further change. The acetonitrile was evaporated under reduced pressure with a rotary evaporator leaving an oily residue. The oily residue was dissolved in 20 ml of sterile water and vaque membrane. The bulk of the water was removed by evaporation and the remainder removed by azeotropic distillation (three times 100 ml toluene) with a rotary evaporator. The residue was precipitated with 200 ml of anhydrous diethylether and dried under vacuum. The weight of the product was 5.18 g. (64.7% by weight).

### Activation

The above amine capped polymer (5.18 g, 287 μmol, Mₙ=18 K, polydispersity=1.35) was dissolved in anhydrous acetonitrile (30 ml) and treated with triethylamine (48.2 mg, 476 μmol) and maleimidopropionic acid NHS ester (Bioscience, 0.2287 g, 859 μmol). The activation reaction was followed until the TLC ninhydrin positive polymer spot became ninhydrin negative. The reaction was found to be complete after 1 hour. The solvent was evaporated and the product purified with 1 L of sterile water in a stirred cell employing a YM-10 membrane The bulk of the water was removed by evaporation and the remainder removed azeotropically (3×30 ml toluene) then vacuum dried for overnight. The product weighted 3.59 g (˜69% by weight).

### Conjugation

Polymer (549 mg, 30.5 μmol) was dissolved in 20 mM phosphate, 5 mM EDTA pH 6.5. sTNF receptor (65.5 mg, 5.4 μmol) was added. The reaction was incubated at 4° C. overnight.

### Purification

The polymer conjugate solution was adjusted to pH 3.5 prior to loading onto the column. The conjugate was purified by FPLC using a Sepharose SP HR column employing 20 mM sodium acetate pH4 and 20 mM sodium acetate plus 0.5 mM NaCl as eluants. Fractions number 70-80 containing free and conjugated sTNF were pooled and concentrated. The pool was loaded onto a SEC 26/60 Sephacryl column S300. The conjugation was collected in two pools as high molecular weight conjugate and low molecular weight conjugate.

To prepare the samples for bioassay, the high and low MW pools were concentrated in Amicon stirred cells (YM-3 membrane) and then each pool was dialyzed against PBS twice (1 L) at 4° C. over a period 24 hrs using 3.5 kDa MWCO membrane. Finally, the samples were concentrated to 2 mg/ml and filtered (25 mm, 0.2 um Acrodicc syringe filters, HT Tuffryn membrane, Gelman laboratory) into 5 ml sterile vials.

### In Vivo Experiments of the Conjugates in Example 5

Soluble TNF-RI conjugates were tested for efficacy in treating a mouse model of inflammation. Lewis rats with collage-induced arthritis (CIA) were injected with conjugates made as described above and termed sTNF/PEG+OXL. The polymer chain consists of 1 kDa PEG molecules linked to.

90 female rates from 80 to 100 g were obtained and arthritis induced as follows. Porcine type II collagen was dissolved in 0.1 N acetic acid (2 mg collagen/ml acetic acid). A 16 gauge emulsification needle was used at 1:1 with adjuvant. Rats were injected intradermally with 100 microliter of emulsion at ten different sites over the dorsal surface of their foot pads.

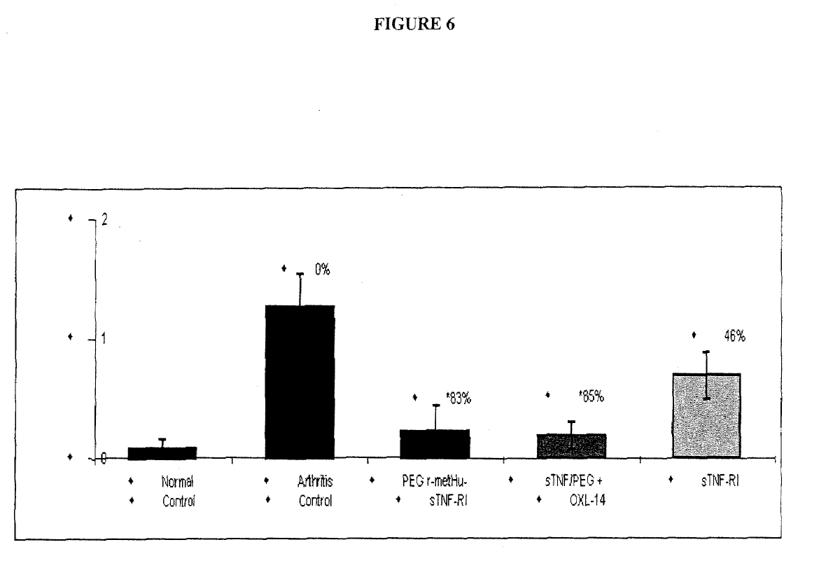

Onset of disease was defined as paw swelling and occurred at day 11. Conjugates were injected at 4 mg/kg subcutaneously starting at onset and was injected daily for three days. There were five groups as normal controls (N=5), untreated controls (N=8), PEG-r-metHu-sTNF-rI (N=8), sTNF/PEG+OXL-14 (N=8), and sTNF-RI (N=8). The arthritis control mice had paw swelling, as measured using calipers daily from onset to conclusion of the study at day three, of about 1 mm at day three (mean +/−SE; n=8). The sTNF-RI treated mice had paw swelling of about 0.5 mm at day three. In contrast, the PEG and PEG+OXL polymers both inhibited paw swelling to the point of being almost undetectable over the three day period (FIG. 6).

While the present invention has been described in terms of preferred embodiments, it is understood that variations and modifications will occur to those skilled in the art. Therefore, it is intended that the appended claims cover all such equivalent variations, which come within the scope of the claims.

What is claimed is:

1. A block polymer comprising a first water soluble polymer of less than 2,000 Daltons, a labile linker, and a second water soluble polymer of less than 2,000 Daltons.

2. The block polymer of claim 1 wherein the water soluble polymer is selected from the group consisting of a polyethylene glycol, a copolymer of ethylene glycol, a copolymer of propylene glycol, a carboxymethylcellulose, a polyvinyl pyrrolidone, a poly-1,3-dioxolane, a poly-1,3,6-trioxane, a ethylene/maleic anhydride copolymer, a polyaminoacids, a dextran n-vinyl pyrrolidone, a poly n-vinyl pyrrolidone, a propylene glycol homopolymer, a propylene oxide polymer, ethylene oxide polymer, a polyoxyethylated polyol and a polyvinyl alcohol.

3. The block polymer of claim 1, wherein the total molecular weight is less than 40 kDa.

4. The block polymer of claim 3, wherein the total molecular weight is about 20 kDa.

5. The block polymer of claim 2 comprising a block copolymer structure.

6. The block polymer of claim 5 comprising a mixture of at least two polymers.

7. The block polymer of claim 2 comprising a block homopolymer structure.

8. The block homo-polymer of claim 7 comprised of blocks of polyethylene glycol.

9. The block polymer of claim 1, wherein 90% of the block polymers are less than 2,000 Daltons.

10. The block polymer of claim 9, wherein the majority of polymer blocks are about 1,000 Daltons.

11. The block polymer of claim 1, wherein the proteolytically sensitive linker comprises an amide bond.

12. A formulation comprising a block polymer of any of claims 1 conjugated to a drug and a pharmaceutically acceptable carrier.

13. A method of making the block polymer of any of claims 1.

14. A method of treating a patient comprising administering a block polymer of any of claims 1.
